(19) United States
(12) Patent Application Publication
(10) Pub. No.: US 2026/0007510 A1
(43) Pub. Date: Jan. 8, 2026

(54) MEDICAL LIFTING THREAD BENT IN ZIGZAG

(71) Applicants: CG BIO CO., LTD., Seoul (KR); M.BASE CO.,LTD, Seongnam-si (KR)

(72) Inventors: Jong Geun KIM, Gwangju-si (KR); Jun Kyu PARK, Seongnam-si (KR); Jin Ho YOO, Seoul (KR)

(73) Assignees: CG BIO CO., LTD., Seoul (KR); M.BASE CO.,LTD, Seongnam-si (KR)

(21) Appl. No.: 19/133,687

(22) PCT Filed: Dec. 1, 2023
(86) PCT No.: PCT/KR2023/019674

§ 371 Date: May 28, 2025

(30) Foreign Application Priority Data
Dec. 6, 2022 (KR) 10-2022-0168408

(51) Int. Cl. A61F 2/10; A61F 2/00

(52) U.S. Cl. CPC A61F 2/105; A61F 2/0077; A61F 2002/0081; A61F 2250/0037

(57) ABSTRACT

A medical lifting thread bent in a zigzag includes a corrugated body on which groove parts and ridge parts, having a height difference, are continuously arranged in a zigzag along the length direction thereof and cogs which are formed on the inner surfaces of the tip portions of the respective groove parts and ridge parts so as to be alternately projected in mutually different directions, and which protrude beyond the adjacent groove or ridge parts. According to the medical lifting thread bent in a zigzag, the effects are achieved whereby the corrugated body formed in a zigzag enables increasing the contact area with biological tissue and securing pliability, and at the same time, the cogs enable preventing extreme curves from forming on the corrugated body while enabling the securing of holding strength, and thus pliability and holding properties may be simultaneously reinforced.

### TECHNICAL FIELD

The disclosure relates to a lifting thread bent in a zigzag shape and, more particularly, to a lifting thread configured to simultaneously improve both flexibility and fixation by means of a body having an overall zigzag shape due to successive repetition of grooves and ridges having a height difference and cogs having a structure capable of achieving fixing force without causing the body to be extremely bent.

### BACKGROUND ART

Medical lifting threads, each having protrusions configured to be inserted into the skin and then hooked into the subcutaneous tissue to pull the skin up, have been widely used by medical practitioners represented by doctors using the so-called thread lifting treatment for the cosmetic purposes of tightening sagging facial skin and reducing nasolabial folds.

Medical lifting threads are typically formed of dissolving thread materials such as polydioxanone (PDO), poly-L-lactic acid (PLLA), and polycaprolactone (PCL) and, based on the dissolving properties of medical lifting threads, retreatment may be performed at regular intervals.

These lifting threads started out as a simple straight body structure, and have evolved into structures including various cogs such as barbs, hooks, cones, and screws to achieve fixing force in the skin tissue.

In particular, lifting threads, each having cogs and groove structures around the cogs to be more firmly fixed to the biological tissue, have been developed. As an example, a medical lifting thread having increased fixing force disclosed in Korean Patent No. 2388216 includes: a body; cogs extending from a surface of the body to be inclined in the rearward direction of the body, and each including a first inclined portion extending from a first point of the body to be inclined in the rearward direction of the body and a second inclined portion extending from an end of the first inclined portion to a second point of the body spaced apart from the first point by a selected distance in the rearward direction of the body; grooves each including a first recessed portion extending into the body on the same line as the second inclined portion and a second recessed portion extending from an end of the first recessed portion to a third point of the body spaced apart from the second point by a selected distance in the rearward direction of the body. A plurality of cogs and a plurality grooves are provided at regular intervals on a first side of the body. The first inclined portion includes a first inclined section extending from the first point of the body to be inclined in the rearward direction of the body and a second inclined section bent at an end of the first inclined section and extending parallel to the direction of extension of the body to be in contact with the second inclined portion. The second recessed portion includes a first recess extending by a selected length from the end of the first inclined section toward a rear end of the body and a second recess bent at the end of the first recess and extending to the third point. A rounded chamfered portion is provided at the boundary between the first recessed portion and the first recess. The structure disclosed in this document may increase fixing force in the biological tissue.

This structure is characterized by the formation of the grooves around the inclined cogs to increase the fixing force of the biological tissue due to the interlocking structure of the cogs and the grooves, but has the problem that the flexibility of the body itself is limited by the straight structure of the body. In addition, because this structure is biased toward an inclined structure having protrusions oriented in a specific direction, this structure is vulnerable to compression from the biological tissue acting in a direction other than the specific direction.

Therefore, it is necessary to develop a lifting thread having a novel and advanced structure that can compensate for the problem of easy breaking by achieving fluidity in the body itself, which has a greater influence on the overall volume and the diameter of the lifting thread than cogs or protrusions, and also increase the fixing force thereof.

### DISCLOSURE

### Technical Problem

The disclosure has been made to overcome the above-described problems, and an objective of the disclosure is to provide a lifting thread configured to simultaneously improve both flexibility and fixation by means of a corrugated body having an overall zigzag shape due to successive repetition of grooves and ridges having a height difference and cogs having a structure capable of achieving fixing force without causing the corrugated body to be extremely bent.

Another objective of the disclosure is to provide a specific structure capable of providing both structural stability and strong fixation of an interlocked structure of the corrugated body and the cogs.

Another objective of the disclosure is to specify gaps between the corrugated body and the cogs to maximize the synergy of intrinsic functions of the corrugated body and the cogs.

Another objective of the disclosure is to achieve the stability of the cogs by a specialized structure of the cogs interlocked with slits.

### Technical Solution

To realize at least one of the above objectives, a lifting thread bent in a zigzag shape according to the disclosure includes: a corrugated body including grooves and ridges having a height difference, which are arranged continuously in a zigzag pattern in a longitudinal direction; and cogs protruding alternately from inner surfaces of tips of the grooves and the ridges in different directions and beyond the adjacent grooves or ridges.

The tips of the grooves and the ridges may include flat portions which are processed to be flat to a selected length, and the cogs may protrude from the flat portions.

Furthermore, the corrugated body may include slopes inclined in converging directions from selected ends of two adjacent grooves of the grooves on opposite sides of a corresponding ridge of the ridges, the slopes having an inclination angle of 5 to 15 degrees relative to the flat portion of the ridge, such that cracks are provided between the slopes and a corresponding cog of the cogs.

### Advantageous Effects

The medical lifting thread bent in a zigzag shape according to the disclosure provides the following effects.

1) The corrugated body having a zigzag shape may increase the contact area with biological tissues and provide flexibility, and the cogs capable of achieving fixing force without causing extreme bending of the corrugated body may simultaneously increase mobility and fixation.

2) The flat portions and the slopes produced by improving pointed portions of the grooves and the ridges may further increase the stability of the corrugated body and the cogs and further improve the fixation of the biological tissue.

3) The cracks, which are the gaps between the corrugated body and the cogs, and the additional recessed slits may increase not only the overall stability of the lifting thread but also strengthen the traction and fixation of the biological tissue.

4) The specialized structure of the slit provides an improved structure of the cogs, which upgrades the upright stability and durability of the cogs and the fixation force with the biological tissue.

### DESCRIPTION OF DRAWINGS

FIG. 1 is a cross-sectional view illustrating an embodiment of a lifting thread of the disclosure.

FIG. 2 is a cross-sectional view illustrating an embodiment in which cogs protrude from flat portions of grooves and ridges.

FIG. 3 is a cross-sectional view illustrating a structure in which slopes are provided on a corrugated body.

FIG. 4 is a cross-sectional view illustrating a structure in which slits are provided on cracks.

FIG. 5 is a cross-sectional view illustrating the structure of cogs formed in conjunction with the slits of the cracks.

FIG. 6 is an enlarged cross-sectional view of part A of FIG. 5.

FIG. 7 is an enlarged cross-sectional view of part B of FIG. 5.

FIG. 8 is a cross-sectional view illustrating a modified embodiment of the cog of FIG. 5.

### BEST MODE

An example configuration for implementing the disclosure includes: a corrugated body including grooves and ridges having a height difference, which are arranged continuously in a zigzag pattern in a longitudinal direction; and cogs protruding alternately from inner surfaces of tips of the grooves and the ridges in different directions and beyond the adjacent grooves or ridges.

### MODE FOR INVENTION

Hereinafter, embodiments of the disclosure will be described in detail with reference to the accompanying drawings. It should be understood that the accompanying drawings may not be drawn to scale, and the same or like reference numerals may be used to refer to the same or like elements throughout the drawings.

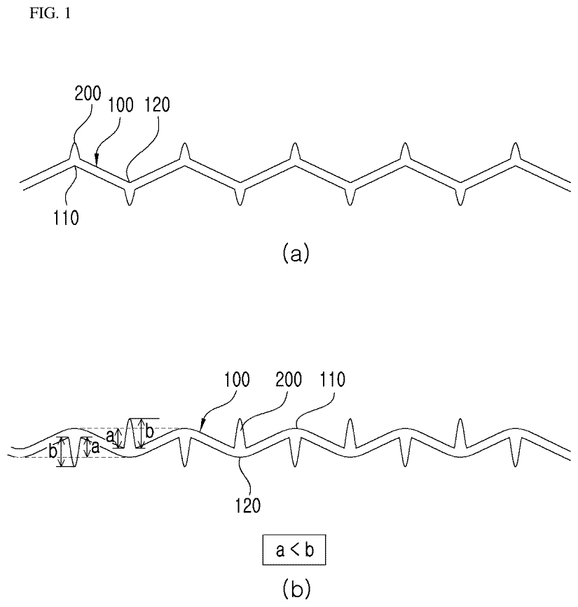

FIG. 1 is a cross-sectional view illustrating an embodiment of a lifting thread of the disclosure.

First, the lifting thread of the disclosure is based on including a body and cogs like a known lifting thread including cogs.

The body may be formed of a material that may dissolve in vivo, such as polydioxanone (PDO), like well-known medical threads, or more desirably, may be formed of or at least include poly-L-lactic (PLLA) or polycaprolactone (PCL), both of which are known as collagen-forming substances.

In particular, the body of the disclosure is based on a zigzag shape unlike the body of the disclosure, and in the disclosure, such a zigzag body is referred to as a corrugated body 100.

The corrugated body 100 of the disclosure has a structure in which grooves 120 and ridges 110 having a height difference are arranged continuously in a zigzag pattern in the longitudinal direction of the corrugated body 100.

Although the corrugated body 100 injected in vivo may not have a vertically arranged structure of different heights as shown in FIG. 1, but for ease of description, the higher portions will be referred to as ridges 110 and the bottom portions will be referred to as grooves 120 with reference to FIG. 1.

As shown in FIGS. 1 (a) and (b), the ridges 110 and the grooves 120 may be formed to maintain the zigzag pattern while having pointed tips, respectively. However, as will be described later, respective portions between the ridges 110 and the grooves 120 may also be gently chamfered without being angled, or the tip of each of the ridges 110 and grooves 120 may be processed into a flat portion 111 or 121 having a selected length.

Based on the length from the front end to the rear end, such a corrugated body 100 may have an increased contact surface area in vivo due to the zigzag shape thereof as compared to a known straight body extending in a straight line, thereby providing increased traction and retention in the in vivo body.

Furthermore, the known straight body may easily break due to natural resilience and repulsive force generated within the biological tissue in which the lifting thread is inserted. In contrast, the corrugated body 100 of the disclosure has the advantage of having the zigzag shape and flexibility, which may efficiently respond to the compressive force of the biological tissue at opposite ends of the corrugated body 100, and thus does not easily break.

To provide an anchor point at which the corrugated body 100 may be securely fixed in vivo, the lifting thread of the disclosure has a plurality of cogs 200 spaced apart from each other by regular intervals on one side of the corrugated body 100, as shown in FIGS. 1 (a) and (b).

The cogs 200 of the disclosure are formed to alternately protrude in different directions from the tips of the grooves 120 and the ridge 110 of the corrugated body 100, respectively, in a state in which the grooves 120 and the ridges 110 are arranged continuously. The lifting thread may be categorized into two structures, as shown in FIGS. 1 (a) and (b), according to the formation positions and the protrusion directions of the cogs 200.

First, opposite sides of the corrugated body 100 of the disclosure (not necessarily two sides because the original corrugated body has a cylindrical shape, but assuming that two sides occur based on the cross-section of FIG. 1), one side of the corrugated body 100 facing away from the corrugated body 100 relative to the tips of the grooves 120 and ridges 110 is defined as the “outer surface”, and the opposite surface facing toward the corrugated body 100 is defined as the “inner surface”.

Referring to FIGS. 1 (a) and (b) based on these definitions, it may be seen that the cogs 200 in FIG. 1 (a) protrude alternately in different directions from the “outer surfaces” of the respective tips of the grooves 120 and the ridges 110, while the cogs 200 in FIG. 1 (b) protrude alternately in different directions from the “inner surfaces” of the respective tips of the grooves 120 and the ridges 110.

In general, it may be understood that the main purpose of the cogs of the lifting thread is to achieve reliable fixing force to the biological tissue by obtaining a plurality of in vivo anchor points. Because the cogs 200 of FIG. 1 (a) protrude in an outward direction relative to the corrugated body 100 (i.e., in the outward direction of the corrugated body), it may be understood that the structure fulfills this purpose.

However, according to the cogs 200 in FIG. 1 (a), the entire lifting thread structure has a number of highly curved S-shaped bends, which causes the problem that opposite ends of the corrugated body 100 do not provide a stable anchorage to the biological tissue and may be severely swayed or distorted by the compression or fluidity of the biological tissue, resulting in instability and inability to perform the intrinsic lifting function.

A representative structure of the disclosure for compensating for these issues is based on the cogs 200 of FIG. 1 (b). The cogs 200 are formed to protrude alternately in different directions from the inner surfaces of the respective tips of the grooves 120 and the ridges 110, which may serve as multi-pronged anchor points due to the extending structure of the cogs 200 while utilizing the bent features of the corrugated body 100 described above.

However, when the cogs 200 of FIG. 1 (b) are buried in areas between the grooves 120 and the ridges 110, adequate fixing force to the surrounding biological tissue may not be properly provided. Accordingly, the cogs 200 of FIG. 1 (b) are desirably formed to protrude higher than the two adjacent grooves 120 or ridges 110 on opposite sides, i.e., to extend further outward from the corrugated body 100 than the tips of the grooves 120 or the ridges 110.

Furthermore, it is desirable that the tips of the groove 120 and the ridges 110 be chamfered rather than pointed to prevent unnecessary bending or flexing under external forces of the biological tissue.

Recapitulating the key features of the lifting thread having the structure of the corrugated body 100 and the cogs 200 of the disclosure, the flexible zigzag shape may efficiently respond to the compression of the biological tissue and may not break easily, while the cogs 200 protrude therebetween (from the inner surface) to provide multiple anchor points, thereby providing a unique characteristic that allows for firm support or anchoring of the biological tissue.

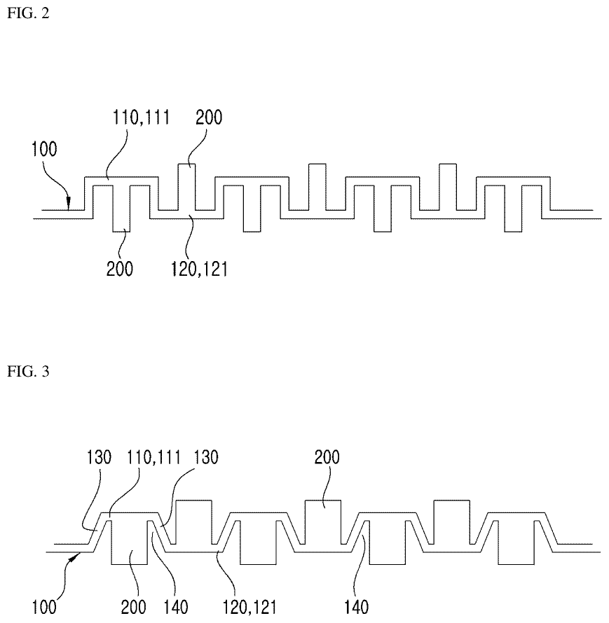

FIG. 2 is a cross-sectional view illustrating an embodiment in which cogs protrude from flat portions of grooves and ridges.

Referring to FIG. 2, it may be seen that the respective tips of the grooves 120 and ridges 110 in the corrugated body 100 are not sharply pointed, but rather flattened to a selected length, and are referred to as “flattened portions” 111 and 121.

The cogs 200 are formed to protrude from these flat portions 111 and 121 of the grooves 120 and ridges 110 in a corresponding manner.

In this case, depending on the greater or lesser height difference between the grooves 120 and the ridges 110, the flat portions 111 and 121 may extend over a length equal to or slightly less than the length of the entire extension of the grooves 120 and the ridges 110. Furthermore, the cogs 200 may also protrude with a width corresponding to the length of the entire extension length of the flat portions 111 and 121, or may protrude with a width that is less than or equal to the entire extension length of the flat portions 111 and 121.

First of all, the reason for achieving the flat portions 111 and 121 by flattening the tip portions is to prevent the problem that the corrugated body 100 is bent into an S-shape while excessively flexing to emphasize flexibility and thus fails to achieve the fixing force at opposite ends as described above. As a result, the corrugated body 100 may be processed to achieve flexibility by bending while maintaining a degree of orientation such as extending in a straight line.

In other words, it is intended to provide an optimal structure that may reliably combine the flexibility and the fixation in vivo of the corrugated body 100.

In this case, the cogs 200 are desirably designed to have a width equal to the length of the flat portions 111 and 121 and to have an appropriately higher protrusion height (e.g., an additional protrusion height of about 0.2 to 0.5 mm) than the adjacent grooves 120 or ridges 110, rather than protruding to an excessively high height, so that the cogs 200 do not cause severe flexing of the lifting thread as a whole, while still achieving the inherent fixing force of the cogs 200.

FIG. 3 is a cross-sectional view illustrating a structure in which slopes are provided on a corrugated body.

FIG. 2 illustrates that the boundary portions of the grooves 120 and the ridges 110 are formed at or near right angles. However, in this case, a structurally unstable arrangement angle (e.g., a right angle) may result at each of the boundary portions between the grooves 120 and the ridges 110 and an unnecessary gap may be formed between the arrangement angles. As a result, the corrugated body 100 may be less responsive to tearing or the appearance of a portion of the corrugated body 100 vulnerable to the repulsive forces of the biological tissue.

To prevent this problem, as may be seen from FIG. 3, the corrugated body 100 desirably includes slopes 130.

Two slopes 130 are inclined relative to a ridge 110 and in converging directions from selected ends of two adjacent grooves 120 on opposite sides of the ridge 110, and may have an inclination angle of 30 to 60 degrees relative to the flat surfaces of the grooves 120 and the flat portion of the ridge 110.

These slopes 130 prevent the grooves 120 and ridges 110 from forming into a vertical structure, which may be referred to as an unstable structure, and at the same time, provide a feature that allows for organic stability between the grooves 120 and ridges 110 while naturally maintaining the flexed structure described above with respect to the overall structure of the corrugated body 100.

At this point, gaps naturally develop between the slopes 130 and the cogs 200, respectively, which are referred to as cracks 140 in the disclosure.

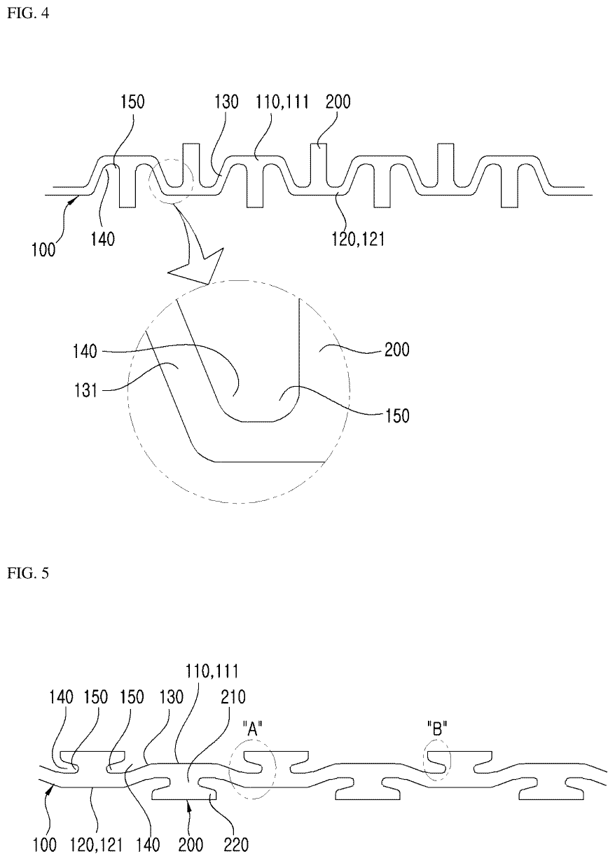

FIG. 4 is a cross-sectional view illustrating a structure in which slits are provided on cracks.

As described above, the cracks 140 are gaps between the slopes 130 and the cogs 200. When the gaps have a pointed shape, such as a “V” shape, the gaps may be structurally vulnerable to external force from the biological tissue and tear away from those areas.

To prevent this problem, each of the cracks 140 includes a slit 150 that is further recessed in the inward direction of the cog 200, more desirably, toward a protrusion start portion of the cog 200 (i.e., an area where the protrusion of the cog 200 starts).

In particular, the slit 150 may not be recessed into the middle portion of the cog 200, but instead may be recessed toward a protrusion start portion of the cog 200, thereby providing features that accommodate a portion of the biological tissue that has penetrated through the crack 140 and into the slit 150 while firmly fixing the biological tissue.

In other words, when the slit 150 is recessed into the central portion of the cog 200, the slit 150 may reduce the upright state of the cog 200, causing the cog 200 to flop around under weak external forces, whereas when the slit 150 is recessed into the protrusion start portion of the cog 200, the cog 200 may advantageously maintain the upright state while reliably achieving an accommodation space for the biological tissue.

Furthermore, each of the slopes 130 may include a recessed rounded portion 131.

As may be seen from FIG. 4, the recessed rounded portion 131 is a portion which extends from the end of the flat portion 111 or 121 to the inner end of the slit 150 to be concavely rounded in the inward direction of the corrugated body 100 (opposite to the protruding direction of the cog).

The recessed rounded portion 131 provides features that may flexibly connect the groove 120 and the ridge 110 to achieve durability while preventing the inclination angle of the slope 130 from forming an unnecessary stepped portion between the groove 120 and the ridge 110. The biological tissue may also be naturally guided toward the slit 150 while uniformly distributing stress to the biological tissue to prevent the corrugated body 100 from breaking due to entrapment of the biological tissue that has begun to be inserted into the slit 150 along the slope 130 or stress concentration in a particular portion of the biological tissue.

That is, in a case in which the slope 130 has a simple straight structure without the recessed rounded portion 131, an inflection point may necessarily occur. Because of a structure in which the inflection point converges toward the corrugated body 100, an action of guiding the biological tissue into the slit 150 may prevent the problem that the durability of the corrugated body 100 is less durable.

FIG. 5 is a cross-sectional view illustrating the structure of cogs formed in conjunction with the slits of the cracks.

Furthermore, two slits 150 on opposite sides of each cog 200 with respect to a protrusion start end of the cog 200 may be symmetrically provided at the same depth of recession. Accordingly, the cog 200 includes an upright portion 210 and a plate 220.

Specifically, the upright portion 210 is a structure protruding between two adjacent (symmetrical) slits and having a first diameter, and the plate 220 is a structure provided on the top end of the upright portion 210, having a second diameter greater than the first diameter of the slit 150, and having a flattened top surface (i.e., the side facing away from the corrugated body).

That is, the cog 200 has a structure protruding in a mushroom-like shape, and the interlocking structure of the slit 150 and the cog 200 has the following advantages.

First, the extension of the slits 150 and the cogs 200 may be formed symmetrically along the entirety of the grooves 120 and the ridges 110 to provide structural stability.

Second, as the cog 200 may be upright between the biological tissue inserted into the two slits 150 symmetrically provided with respect to the cog 200 so that the biological tissue may be spread apart due to the plate 220. As a result, the problem that the biological tissue is excessively inserted into the slits 150 and causes the corrugated body 100 to easily break or fracture may be prevented.

Third, a balance of opposing forces may be achieved between the flexibility of the zigzag shape of the corrugated body 100 and the fixation of the cogs 200.

Finally, the plate 220 of the COG 200 protruding in a manner that covers the entrance of the slit 150 without being oriented in a particular direction may not only increase the durability of the biological tissue to external forces acting in multiple directions, but also provide the ability to better control the amount of the biological tissue inserted into the slit 150 and further prevent unnecessary bending of the corrugated body 100 by the biological tissue as compared to the structure in which the entrance of the slit 150 is open.

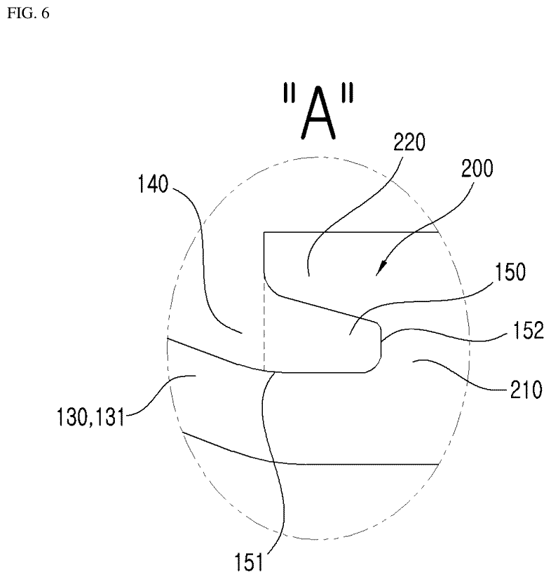

FIG. 6 is an enlarged cross-sectional view of part A of FIG. 5.

Referring to FIG. 6, the slit 150 is shown as having an advanced structure rather than being a typical rounded groove. The slit 150 includes an expansion rounded portion 151 that is a lower extension of the slit 150 and an extended portion 152 that is an upper extension of the slit 150 with respect to the cross-sectional structure of FIG. 5.

Specifically, the expansion rounded portion 151 refers to a portion that extends from an end of the slope 130 to a protrusion start point of the upright portion 210 to be concavely rounded in the inward direction of the corrugated body 100.

The expansion rounded portion 151 may exist independently of the recessed rounded portion 131 described above, but in an example embodiment, the expansion rounded portion 151 may also interlock with the recessed rounded portion 131 such that the recessed rounded portion 131 and the expansion rounded portion 151 extend smoothly without a significant stepped portion.

Like the recessed rounded portion 131 of the slope 130, the expansion rounded portion 151 serves to prevent an unnecessary stepped portion from being formed in the slit 150 while also preventing the cog 200 from breaking due to the entrapment of the biological tissue in the slit 150 or stress concentrated in a particular area of the biological tissue.

The extended portion 152 extends from the expansion rounded portion 151, past the upright portion 210, to a side portion of the plate 220, which is the portion that defines the contour of the cog 200. The extended portion 152 may be specifically specialized as follows.

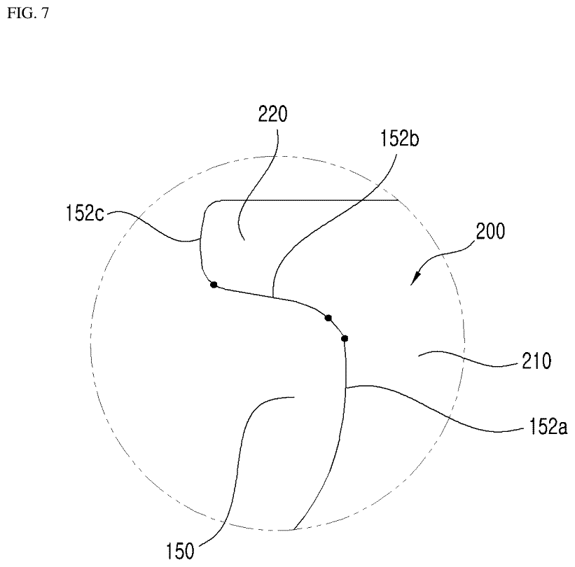

FIG. 7 is an enlarged cross-sectional view of part B of FIG. 5.

As may be seen from FIG. 7, the extended portion 152, which defines the contour of the cog 200 while serving as the upper extension of the slit 150, desirably includes first, second, and third sections 152a, 152b, and 152c.

The first section 152A is a portion that extends from an end of the expansion rounded portion 151 (i.e., the start point of the extended portion) to the boundary between the upright portion 210 of the cog 200 and the bottom portion of the plate 220 to be concavely rounded in the central direction of the upright portion 210. In this case, the central direction of the upright portion 210 refers to a direction toward the interior of the upright portion 210 having a cylindrical shape.

The second section 152b is also a portion that extends from an end of the first section 152a along a low surface extension of the plate 220 to be concavely rounded in the direction of the top surface of the plate 220. Here, the direction of the top surface of the plate 220 refers to a direction toward the flat top surface of the plate 220 with respect to FIG. 7.

The third section 152c is a portion that extends from an end of the second section 152b to the top end of a side portion of the plate 220 to be convexly rounded outward from the side portion of the plate 220. Here, assuming that the side portion of the plate 220 forms a vertical line of the longitudinal axis with respect to FIG. 7, the outward direction of the side portion of the plate 220 refers to a direction toward the outside rather than toward the inside of the plate 220 on this perpendicular line.

The first, second, and third sections 152A, 152B, and 152C provide the upper extension of the slit 150, i.e., the extended portion 152, which defines the contour of the COG 200, with a rounded shape to prevent the occurrence of an angled stepped portion.

Specifically, the first section 152a may cooperate with the expansion rounded portion 151 to prevent an angled portion from being formed at the tip of the slit 150, thereby reducing tearing of the upright portion 210 of the cog 200 due to the angled portion, and may be inserted into the upright portion 210 to provide a base for the upright portion 210 to remain resiliently upright.

In addition, the second section 152b is concavely rounded in the upward direction of the plate 220 to prevent the peripheral side of the plate 220 from sagging toward the corrugated body 100 and to provide a base for the plate 220 to stably maintain the circular shape while being tensioned.

In addition, the third section 152c extends convexly outward from the plate 220 and serves to prevent the plate 220 from being stuck between biological tissues, in which case the plate 220 would be easily crushed and fail to maintain the unique shape thereof.

The upper extension of the slit 150, or the extended portion 152, may be constructed of the first, second, and third sections 152a, 152b, and 152c to be characterized by not only strengthening the function of the slit 150 itself but also providing a base for the cog 200 to have a more stable structure.

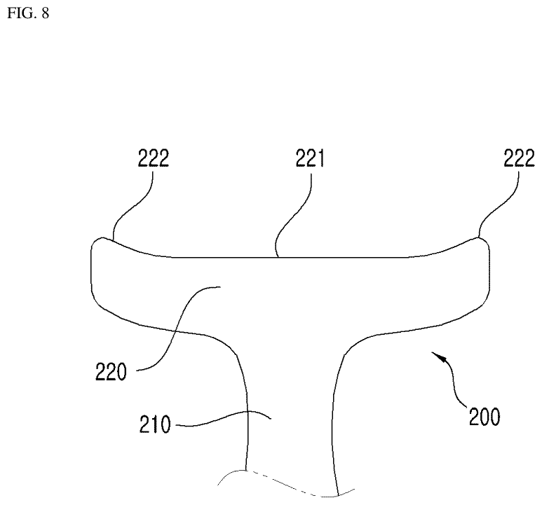

FIG. 8 is a cross-sectional view illustrating a modified embodiment of the cog of FIG. 5.

FIG. 8 is directed to a specialized structure of the top surface of the plate 220, specifically including a flat section 221 and bent sections 222.

The flat section 221 is a portion that extends from the central portion of the plate 220 in a circumferential direction to have a selected area, and the bent sections 222 are portions that extend upward from ends of the flat section 221 to be rounded in the opposite direction of the slit 150.

As used herein, “extending upward” means that the bent sections 222 extend outward, in a bent state, away from the corrugated body 100.

That is, due to the bent sections 222, the top surface of the plate 220 has a dome or basin structure around the flat section in the central portion.

According to this structure, the problem of the top surface of the plate 220 sagging toward the corrugated body 100 due to the upright portion 210 having the first diameter smaller than the diameter of the plate 220 (i.e., the second diameter) may be prevented, thereby achieving the advantage of reliably maintaining the uprightness of the cog 200. This structure may also provide a feature that the top surface of the plate 220 may adhere to the surrounding biological tissue as if adsorbing the biological tissue, thereby providing a strong adhesion with the biological tissue while causing the peripheral portion of the biological tissue to be inserted toward the slit 150, thereby achieving a fixing force based on the strong adhesion with the biological tissue around the cog 200.

The configurations and functions of the lifting thread bent in a zigzag shape according to the disclosure have been described with reference to the drawings. It should be understood, however, that the foregoing descriptions are illustrative only, and the technical idea of the disclosure is not limited to the foregoing descriptions or the accompanying drawings. Those having ordinary knowledge in the art will appreciate that various modifications and changes in forms are possible without departing from technical idea of the disclosure.

### INDUSTRIAL APPLICABILITY

The disclosure is considered industrially applicable because mass production using industrial facilities is possible.

1. A lifting thread bent in a zigzag shape, the lifting thread comprising:
a corrugated body comprising grooves and ridges having a height difference, which are arranged continuously in a zigzag pattern in a longitudinal direction; and
cogs protruding alternately from inner surfaces of tips of the grooves and the ridges in different directions and beyond the adjacent grooves or ridges.

2. The lifting thread of claim 1, wherein the tips of the grooves and the ridges comprise flat portions which are processed to be flat to a selected length, and
the cogs protrude from the flat portions.

3. The lifting thread of claim 2, wherein the corrugated body comprises slopes inclined in converging directions from selected ends of two adjacent grooves of the grooves on opposite sides of a corresponding ridge of the ridges, the slopes having an inclination angle of 5 to 15 degrees relative to the flat portion of the ridge,
such that cracks are provided between the slopes and a corresponding cog of the cogs.

4. The lifting thread of claim 3, wherein the cracks comprise slits, respectively, which are further recessed in an inward direction toward a protrusion start portion of the cog.

5. The lifting thread of claim 4, wherein each of the slopes comprises a recessed rounded portion extending from an end of the flat portion to a start end of the slit to be concavely rounded in an inward direction of the corrugated body.

6. The lifting thread of claim 4, wherein the slits are provided symmetrically with respect to the protrusion start portion of the cog and have an equal depth of recession, and
the cog comprises:
an upright portion protruding between the two adjacent slits and having a first diameter; and
a plate provided on a top end of the upright portion, having a second diameter greater than the first diameter, and having a flattened upper surface.

7. The lifting thread of claim 6, wherein each of the slits comprises:
an expansion rounded portion extending from an end of the slope to a protrusion start point of the upright portion to be concavely rounded in an inward direction of the corrugated body; and
an extended portion extending from an end of the expansion rounded portion, past the upright portion, to a side portion of the plate.

8. The lifting thread of claim 7, wherein the extended portion comprises:
a first section extending from the end of the expansion rounded portion to a boundary between the upright portion and a bottom portion of the plate to be concavely rounded in a central direction of the upright portion;
a second section extending from an end of the first section a boundary between the bottom portion and the side portion of the plate to be concavely rounded in a direction of a top surface of the plate; and
a third section extending from an end of the second section to a top end of the side portion of the plate to be convexly rounded outward from the side portion of the plate.

9. The lifting thread of claim 8, wherein the top surface of the plate comprises:
a flat section extending from a central portion of the plate in a circumferential direction to have a selected area; and
bent sections extending from ends of the flat section in an opposite direction of the slit to be rounded upward.
